(19) United States
(12) Patent Application Publication
(10) Pub. No.: US 2022/0193298 A1
(43) Pub. Date: Jun. 23, 2022

(54) APPARATUS AND METHOD FOR ULTRAVIOLET DISINFECTION

(71) Applicant: Blu Technology LLC, Portage, MI (US)

(72) Inventors: Corbin Collet, Portage, MI (US); April Jennesis, Kalamazoo, MI (US); Brian Brandon, Bellevue, MI (US)

(21) Appl. No.: 17/554,885

(22) Filed: Dec. 17, 2021

Related U.S. Application Data
(60) Provisional application No. 63/127,259, filed on Dec. 18, 2020.

(51) Int. Cl. A61L 9/20; A61L 2/10

(52) U.S. Cl. CPC A61L 9/20; A61L 2/10; A61L 2202/11; A61L 2202/25; A61L 2202/14; A61L 2209/111; A61L 2209/12

(57) ABSTRACT

An antimicrobial system for disinfecting surrounding surfaces and air passing through an apparatus mounted above a space to be disinfected. The system includes a disinfecting apparatus that includes an ultraviolet source emitting ultraviolet light for disinfecting exposed surfaces. The system also includes a fixture module retaining and supporting the ultraviolet source within the disinfecting apparatus. The system includes a drive module that positions the ultraviolet source in one of two positions within the fixture module. The ultraviolet source emits ultraviolet light onto exposed surfaces when in a “ready” position within the fixture module. The ultraviolet source is shielded when in a “storage” position within the fixture module, such that emitted ultraviolet light will not reach the exposed surfaces. The disinfecting apparatus is configured to disinfect air passing through the disinfecting apparatus when the ultraviolet source is in the “storage” position.

### CROSS REFERENCE TO RELATED APPLICATIONS

The present application claims the filing benefits of U.S. provisional application, Ser. No. 63/127,259, filed Dec. 18, 2020, which is hereby incorporated by reference herein in its entirety.

### FIELD OF THE INVENTION

The present invention is directed to ultraviolet sanitation, and in particular, to using ultraviolet light to disinfect air and surfaces.

### BACKGROUND OF THE INVENTION

Conventional ultraviolet light sources, such as ultraviolet C (UV-C) light bulbs or light fixtures, have been used to disinfect surfaces. UV-C wavelengths typically used for disinfecting include, for example, 222 nm and 254 nm. UV light sources are usually positioned so that when energized, they emit ultraviolet light to disinfect surfaces exposed to the ultraviolet light.

### SUMMARY OF THE INVENTION

Embodiments of the present invention provide a system, apparatus, and methods for disinfecting a space (e.g., positioning an apparatus above a space to be disinfected), as well as for disinfecting air forced through the apparatus. An apparatus positioned above a space to be disinfected is provided for disinfecting that space and for disinfecting air passing through the apparatus. The apparatus includes an ultraviolet source configured to emit germicidal ultraviolet light. The ultraviolet source is configured to emit ultraviolet light onto surrounding surfaces of the space when in a first position. The ultraviolet source is configured to disinfect air passing through the apparatus when the ultraviolet source is in a second position. The ultraviolet source, when in the second position, does not emit ultraviolet light onto the surrounding surfaces. The apparatus includes a fixture module for moving the ultraviolet source between the first position and the second position.

In an aspect of the present invention, an antimicrobial system is provided for disinfecting surrounding surfaces of a space and for disinfecting air passing through a disinfecting apparatus. The system includes a disinfecting apparatus that includes an ultraviolet source operable to emit ultraviolet light operable to disinfect exposed surfaces of the space. The space is a surrounding area or room. The disinfecting apparatus includes a fixture module configured to retain and support the ultraviolet source within the disinfecting apparatus. The disinfecting apparatus also includes a drive module configured to position the fixture module and the ultraviolet source in one of two positions. The ultraviolet source is configured to emit ultraviolet light onto exposed surfaces of the space when in a “ready” position within the fixture module. The ultraviolet source is shielded from the space when in a “storage” position within the fixture module such that emitted ultraviolet light will not reach the exposed surfaces of the space. The disinfecting apparatus is configured to disinfect air passing through the disinfecting apparatus when the ultraviolet source is in the second position.

In another aspect of the present invention, a method for disinfecting surrounding surfaces of a space and for disinfecting air passing through a disinfecting apparatus includes emitting ultraviolet light from an ultraviolet source mounted above the space that is operable to disinfect exposed surfaces of the space. The space is a surrounding area or room. The ultraviolet source is placed into a “ready” position such that the emitted ultraviolet light can reach exposed surfaces of the space. The ultraviolet source is placed into a “storage” position such that emitted ultraviolet light will not reach the exposed surfaces of the space. The method also includes drawing air into the disinfecting apparatus while the ultraviolet source is in the “storage” position such that the drawn air is passed through the ultraviolet light to disinfect the air.

In an aspect of the present invention, the ultraviolet source is configured to emit ultraviolet C (UV-C) light.

In another aspect of the present invention, the air passing through the apparatus, and through the ultraviolet light, is fan forced. When the ultraviolet source is in the “storage” position, the fan-forced air is passed through ultraviolet light emitted by the ultraviolet source.

In a further aspect of the present invention, when the ultraviolet source is in the “ready” position, the ultraviolet source has been repositioned such that the emitted ultraviolet light reaches the surrounding area. Repositioning the ultraviolet source may include one of: rotating the ultraviolet source along a horizontal axis, rotating an end of the ultraviolet source down into a room from a ceiling mounted position, and removing a cover over the ultraviolet source.

In yet another aspect of the present invention, the ultraviolet source may include one or more ultraviolet light sources, including one or more 222 nm or 254 nm incandescent light fixtures, 222 nm or 254 fluorescent light fixtures, 222 nm excimer light fixtures, or 254 nm light emitting diodes (LEDs).

Thus, an apparatus containing an ultraviolet source may be mounted above a space to be disinfect, with the ultraviolet source used to disinfect exposed surfaces of the space, which may include a surrounding area or room. The ultraviolet source may also be used to disinfect air that passes through the apparatus when the ultraviolet source is in a “storage” position within the apparatus and not actively disinfecting the exposed surfaces of the space below the apparatus.

These and other objects, advantages, purposes, and features of the present invention will become apparent upon review of the following specification in conjunction with the drawings.

### BRIEF DESCRIPTION OF THE DRAWINGS

FIG. 1 is a block diagram of an exemplary apparatus for disinfecting surfaces that is positioned within a troffer light fixture in accordance with the present invention;

FIG. 2A is a perspective view of an exemplary disinfecting apparatus with air intake and exhaust, and illustrating an ultraviolet source extending from one side of the apparatus in accordance with the present invention;

FIG. 2B is a perspective view of the disinfecting apparatus of FIG. 2A configured as a stand-alone unit in accordance with the present invention;

FIG. 3 is a perspective view of another disinfecting apparatus without air intake and exhaust, and illustrating an ultraviolet source extending from one side of the apparatus in accordance with the present invention;

FIG. 4 is a side view of the disinfecting apparatus of FIG. 2A, 2B, or 3 and illustrating the rotating of the ultraviolet source to raise or lower the ultraviolet source in accordance with the present invention;

FIG. 5 is a perspective view of another exemplary disinfecting apparatus with air intake and exhaust in accordance with the present invention;

FIGS. 6A and 6B illustrate perspective views of another exemplary disinfecting apparatus with air intake and exhaust, and illustrate an ultraviolet source that rotates around a longitudinal axis in accordance with the present invention;

FIGS. 7A and 7B illustrate perspective views of yet another exemplary disinfecting apparatus with air intake and exhaust, and illustrate an ultraviolet source that rotates around a longitudinal axis in accordance with the present invention;

FIG. 8 illustrates a perspective view of another exemplary disinfecting apparatus with air intake and exhaust, and arranged within a troffer light fixture in accordance with the present invention;

FIG. 9 illustrates a perspective view of an additional exemplary disinfecting apparatus arranged within a troffer light fixture, and illustrates an ultraviolet source that rotates around a longitudinal axis within a housing of the troffer light fixture in accordance with the present invention;

FIGS. 10A-10H illustrate perspective views of a plurality of different disinfecting apparatuses arranged within troffer light fixtures in accordance with the present invention;

FIG. 11 is a cross-sectional view of an exemplary disinfecting apparatus illustrating vent arrangement and the passage of air through the antimicrobial apparatus and over an ultraviolet source that has been rotated about a longitudinal axis in accordance with the present invention;

FIG. 12 is a block diagram of an exemplary disinfecting system illustrating a control module communicatively coupled to an occupancy sensor for controlling the operation of one or more disinfecting apparatuses in accordance with the present invention;

FIGS. 13A and 13B illustrate perspective views of a disinfecting apparatus arranged within a troffer light fixture in accordance with the present invention;

FIGS. 14A and 14B illustrate additional perspective views of the disinfecting apparatus of FIGS. 13A and 13B;

FIGS. 15A and 15B illustrate perspective interior views of the disinfecting apparatus of FIGS. 13A and 13B in accordance with the present invention;

FIGS. 16A and 16B illustrate additional perspective views of the disinfecting apparatus of FIGS. 13A and 13B and illustrating the lowering of an ultraviolet source in accordance with the present invention;

FIGS. 17A and 17B illustrate additional perspective interior views of the disinfecting apparatus of FIGS. 13A and 13B and illustrating the lowering of an ultraviolet source in accordance with the present invention;

FIGS. 18A and 18B illustrate side views of the disinfecting apparatus of FIG. 17A and further illustrating the lowering of an ultraviolet source in accordance with the present invention;

FIGS. 19A-19C illustrate perspective views of an ultraviolet source in accordance with the present invention;

FIGS. 20A-20B illustrate diagrams of a side view of the ultraviolet source of FIGS. 19A-19C;

FIG. 21A illustrates a perspective view of a disinfecting apparatus illustrating a rotatable ultraviolet fixture rotated to expose traditional lighting sources;

FIG. 21B illustrates a perspective view of the disinfecting apparatus of FIG. 21A illustrating the rotatable ultraviolet fixture rotated to expose ultraviolet sources opposite the traditional lighting sources;

FIG. 22A illustrates a perspective view of the disinfecting apparatus of FIG. 21A and illustrating the traditional lighting sources energized;

FIG. 22B illustrates a perspective view of the disinfecting apparatus of FIG. 21B and illustrating the ultraviolet sources energized as well as illustrating energized tell-tale lights;

FIG. 23A illustrates another perspective view of the disinfecting apparatus of FIG. 22B; and

FIG. 23B illustrates another perspective view of the disinfecting apparatus of FIG. 22A.

### DESCRIPTION OF THE PREFERRED EMBODIMENTS

Referring to the drawings and the illustrative embodiments depicted therein, a disinfecting apparatus with an ultraviolet source is mounted above a space to be disinfected and is used to disinfect the exposed surfaces of that space, as well as used to disinfect air that passes through the apparatus when the ultraviolet source is in a “storage” position within the apparatus, and not actively disinfecting the exposed surfaces of the space. Note that the space may be a surrounding area or room. The ultraviolet source is configured to emit ultraviolet light, e.g., ultraviolet C (UV-C) light, which disinfects those surfaces that are reached by the ultraviolet light. The apparatus may comprise one or more ultraviolet light sources, with the ultraviolet light provided by incandescent bulbs or light fixtures, excimer light fixtures, and light-emitting diodes (LEDs), as well as any other light fixture capable of emitting ultraviolet light. Because the disinfecting apparatus is configured to be mounted above a space to be disinfected, the disinfecting apparatus may be configured for mounting on a ceiling of a room, mounted within an archway, suspended over the desired space to be disinfected, mounted on or near a ceiling of a mode of transportation (e.g., a taxi, bus, train, and airplane). As also discussed herein, the disinfecting apparatus may be incorporated into troffer lighting fixtures or configured as standalone units.

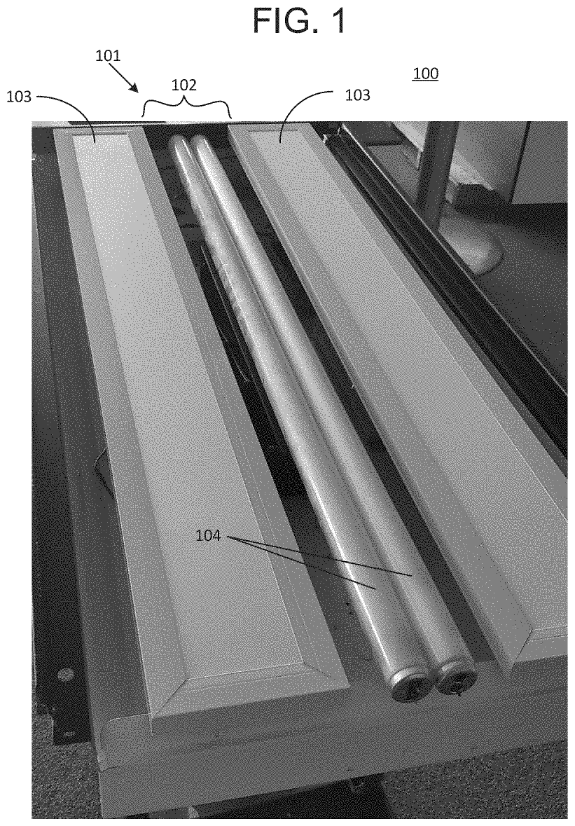

FIG. 1 illustrates a troffer light fixture 100. Troffer light fixtures are rectangular light fixtures configured for insertion into modular dropped ceiling grids or other similar ceiling arrangements. Alternatively, troffer light fixtures may be configured as surface-mounted light boxes. Troffer light fixtures are commonly equipped with fluorescent lamps, incandescent light fixtures, or LED light fixtures. The troffer light fixture 100 of FIG. 1 includes a disinfecting apparatus 102 and a pair of light panels 103 which may contain one or more of types of light fixtures. Those light fixtures may include fluorescent lighting, incandescent lighting, and LED lighting. An exemplary disinfecting apparatus 102 can comprise one or more ultraviolet sources 104 configured to emit ultraviolet C (UV-C) light in a selected disinfecting or germicidal wavelength ranging from 222 nm to 280 nm, e.g., 222 nm and 254 nm. As illustrated in FIG. 1, the ultraviolet source 104 is retained and supported within the disinfecting apparatus 102 by a fixture module 101. The ultraviolet source 104 is energized when it receives power through a wiring harness that is arranged within the fixture module 101. The ultraviolet source 104 is energized with any of a variety of different power inputs, for example, 12 VDC, 24 VDC, 120 VAC, and 240 VAC. As discussed herein, the disinfecting apparatus 102 is configured to control the operation of the ultraviolet source 104 via the wiring harness of the fixture module 101.

As illustrated in FIG. 1, the ultraviolet source 104 can comprise a pair of linear UV-C bulbs/lighting fixtures or a pair of linear arrays of UV-C bulbs/lighting fixtures. Alternatively, the ultraviolet source 104 can comprise a single linear bulb/light fixture or a linear array of bulbs/lighting fixtures.

The ultraviolet source 104 may comprise one or more ultraviolet light fixtures, such as one or more fluorescent light fixtures, arrays of light-emitting diodes (LEDs), excimer light fixtures, or incandescent light fixtures, which may be arranged as one or more linear ultraviolet light fixtures or arranged in linear arrays of ultraviolet light fixtures. UV-C LEDs and UV-C incandescent light fixtures may be configured to emit ultraviolet light at a wavelength of 254 nm. UV-C excimer light fixtures may be configured to emit ultraviolet light at a wavelength of 222 nm. Meanwhile, UV-C fluorescent light fixtures may be configured to emit ultraviolet light at a wavelength of 222 nm or 254 nm. Other wavelengths within the ultraviolet C light range used for ultraviolet antimicrobial treatment are also possible. For example, UV-C wavelengths are those wavelengths between 100 nm to 280 nm, such as those wavelengths between 222 nm to 280 nm. Those selected wavelengths discussed above are particularly germicidal in that they damage the DNA of microorganisms (e.g., bacterium, viruses, and other pathogens) which prevents the microorganisms from reproducing.

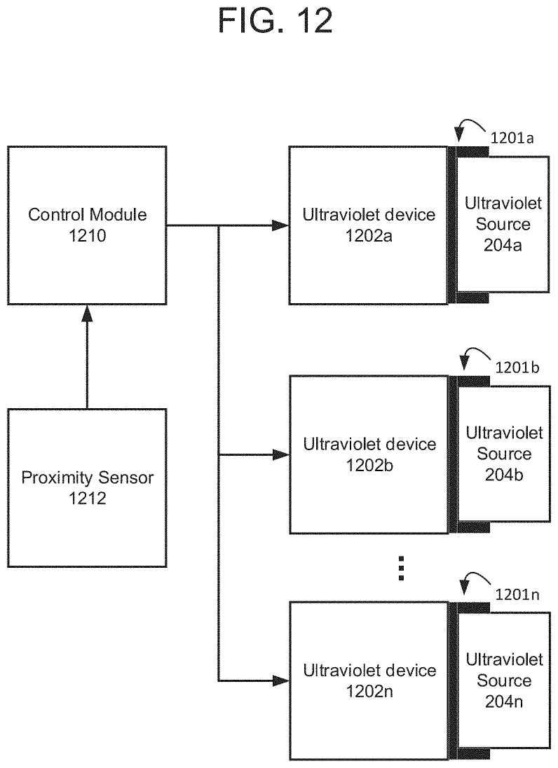

When one or more troffer light fixtures 100 (each containing a respective disinfecting apparatus 102) are positioned within a room or area, energizing the disinfecting apparatuses 102 will expose surfaces within the room or area to the combined ultraviolet light emitted by their respective ultraviolet sources 104. The troffer light fixtures 100 with corresponding disinfecting apparatuses 102 may be arranged within a ceiling grid to provide an even coverage of ultraviolet light to the surrounding area or room. FIG. 12 illustrates the control of an arrangement of disinfecting apparatuses 1202a-n by a control module 1210 communicatively coupled to a proximity sensor 1212.

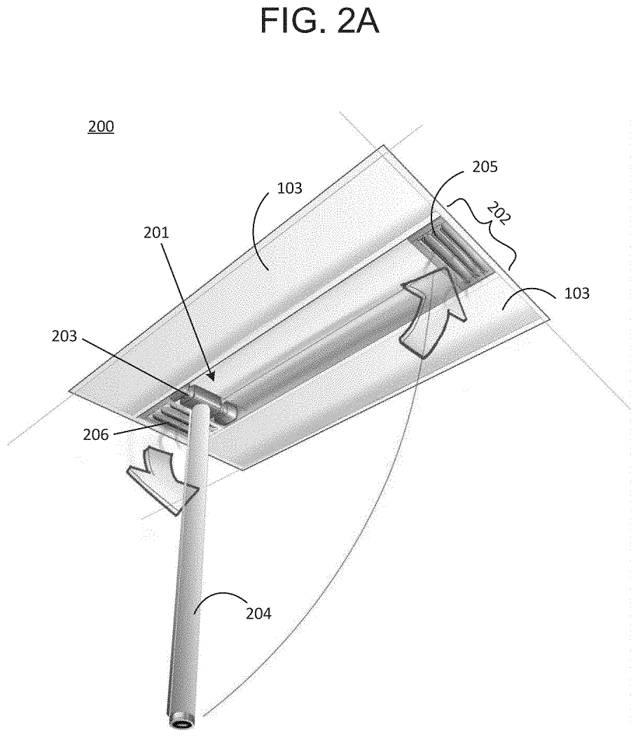

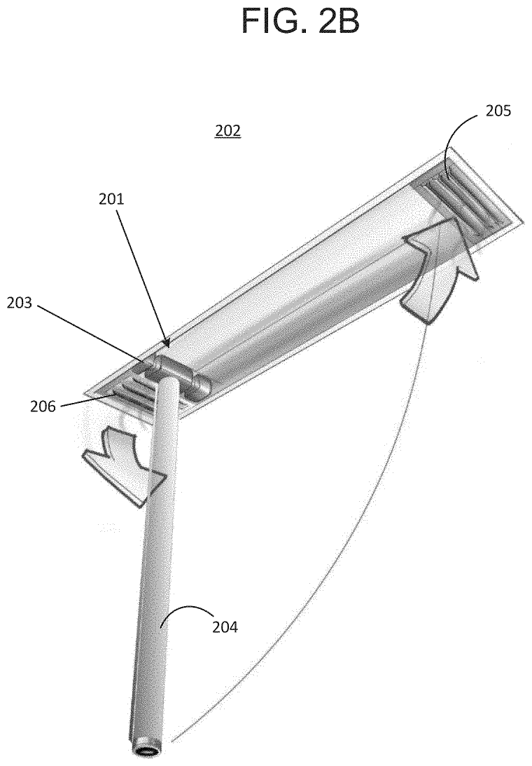

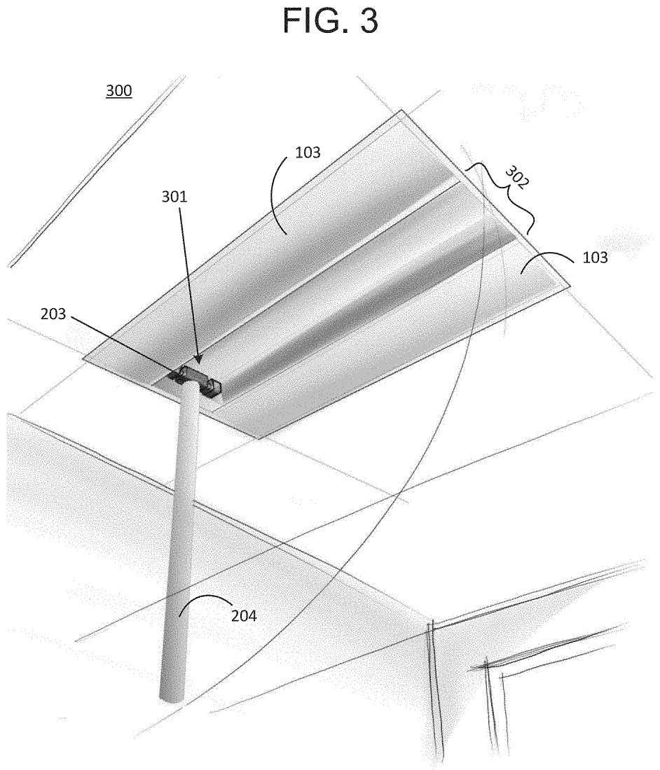

FIGS. 2A and 3 illustrate troffer light fixtures (such as illustrated in FIG. 1) with deployable disinfecting apparatuses 202, 302. The disinfecting apparatus 202 of FIG. 2A also includes a pair of air vents 205, 206, which are described in detail below. FIG. 2B illustrates an alternative version of the disinfecting apparatus 202 of FIG. 2A, which is configured as a standalone unit. When configured as a standalone unit, a plurality of the disinfecting apparatuses 202 may be arranged above a space to be disinfected (above a surrounding area or room to be disinfected). In a further alternative, a combination of standalone disinfecting apparatuses 202 and those incorporated into troffer light fixtures may be arranged on the ceiling of an area or room, with the standalone disinfecting apparatuses 202 not necessary mounted to the ceiling (alternative mounting means an locations may be utilized).

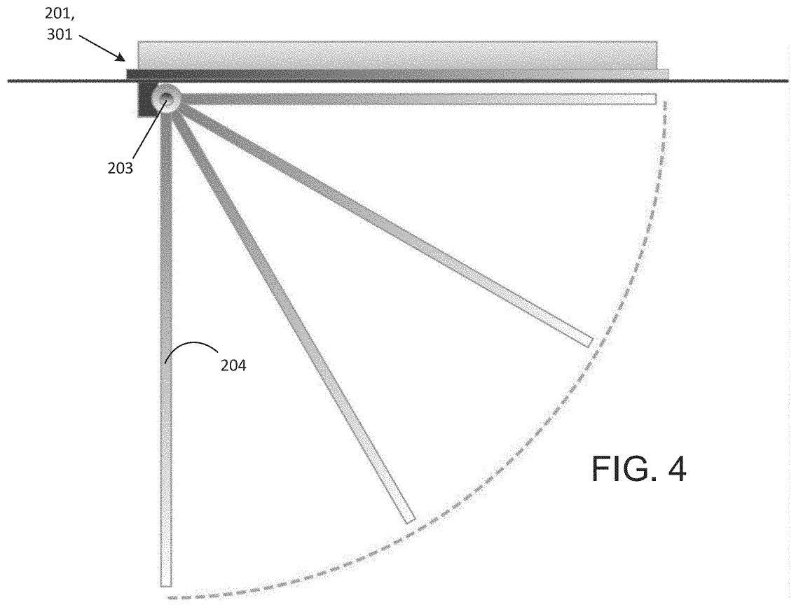

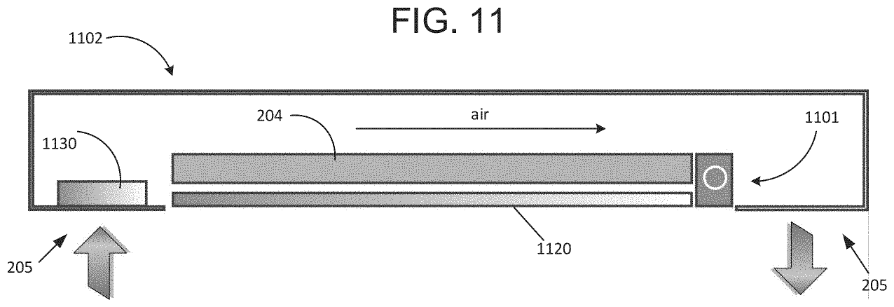

As illustrated in FIG. 4, the disinfecting apparatuses 202, 302 of FIGS. 2A, 2B, and 3 are considered “deployable” in that they are each configured to rotate an ultraviolet source 204 (that is fastened on one end to a hinge 203) from a “ready” position in the disinfecting apparatus 202, 302 to a “deployed” position, such that the ultraviolet source 204 extends perpendicularly from the disinfecting apparatus 202, 302 (see FIGS. 2A and 3). The hinges 203 and ultraviolet sources 204 are retained and supported within respective fixture modules 201, 301 of the corresponding disinfecting apparatuses 202, 302. Note that while in the “ready” position (see FIG. 5), the ultraviolet source 204 is facing toward the room or area but not yet rotated to extend into the room or area. FIGS. 2A, 2B, and 3 illustrate the disinfecting apparatuses 202, 302 with their respective ultraviolet sources 204 in the “deployed” position. When rotating about the hinge 203 to extend from the disinfecting apparatus, the ultraviolet source 204 rotates or extends down and into the room, such that the emitted ultraviolet light evenly reaches the exposed horizontal and vertical surfaces in the room. Necessary hardware (e.g., a motor and associated gearing) to raise and lower the ultraviolet source 204 is also retained and supported by the fixture module 201, 301 (see FIG. 11).

FIG. 12 illustrates a block diagram of a disinfecting antimicrobial system 1200 that includes a control module 1210 for controlling the operation of one or more disinfecting apparatuses 1202a-n arranged to provide ultraviolet sanitation of exposed surfaces of an area or room. As controlled by the control module 1210, the disinfecting apparatuses 1202a-n are instructed to enter a “disinfect” mode and to emit ultraviolet light so long as a proximity sensor 1212 of the system 1200 does not detect the presence of a person. When a person is detected by the proximity sensor 1212, the control module 1210 will not allow the disinfecting apparatuses 1202a-n to enter the disinfect mode, and will instruct the disinfecting apparatuses 1202a-n to cease from a current disinfect mode and enter a “standby” mode where the disinfecting apparatuses 1202a-n are not emitting ultraviolet light into the room or area monitored by the proximity sensor 1212.

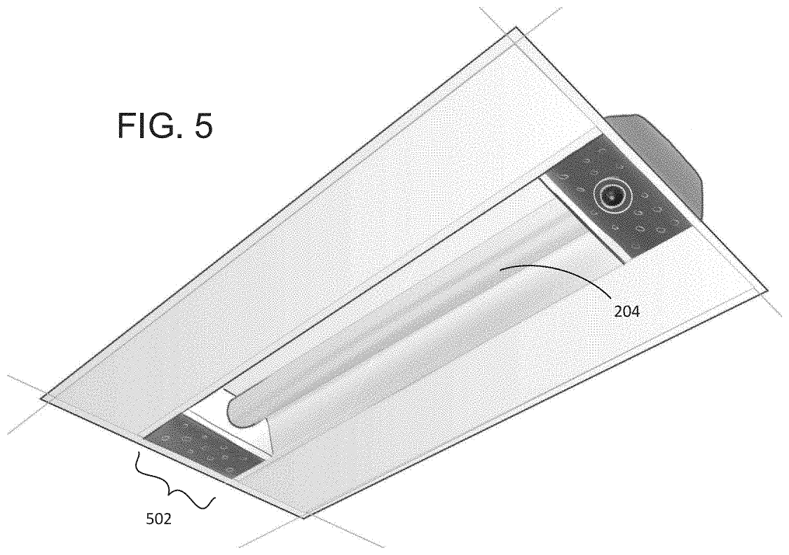

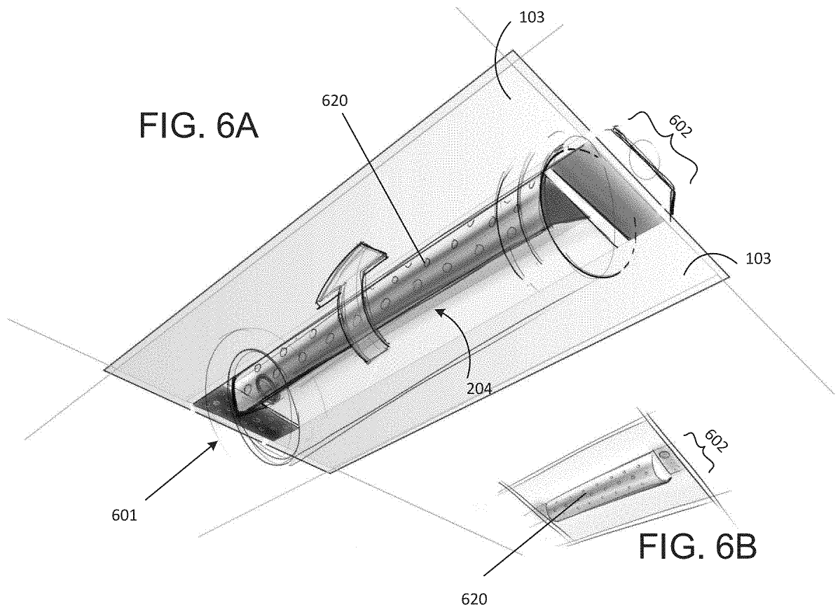

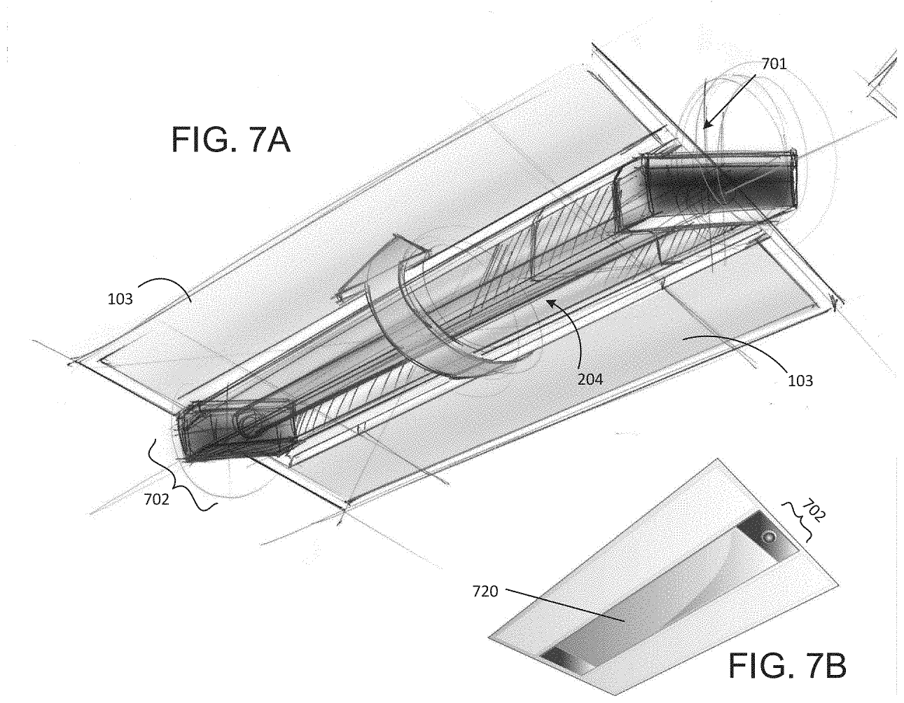

FIGS. 6A, 6B, 7A, 7B, and 9 illustrate exemplary rotatable disinfecting apparatuses 602, 702, and 902, with each disinfecting apparatus comprising a corresponding fixture module 601, 701, and 901 configured to rotate their respective ultraviolet sources 204 about a longitudinal axis. For example, when the fixture modules 601, 701 have rotated such that their respective ultraviolet sources 204 have rotated about their longitudinal axes to face the surrounding area or room, the ultraviolet sources 204 are in the “ready” position (see FIGS. 6A and 7A, where the fixture modules 601, 701 are rotating to place their respective ultraviolet sources 204 into the “ready” position). The fixture modules 601, 701 may also be rotated to rotate their ultraviolet sources 204 about their longitudinal axes to face into their respective disinfecting apparatus 602, 702, such that their ultraviolet sources 204 are in the “storage” position (see FIGS. 6B, 7B, and 8 where the ultraviolet sources 204 (via their respective fixture modules 601, 701) have been rotated such that they are facing away from the room or area and into their respective disinfecting apparatuses 602, 702, and 802, such that back covers 620, 720, and 820 are now visible.

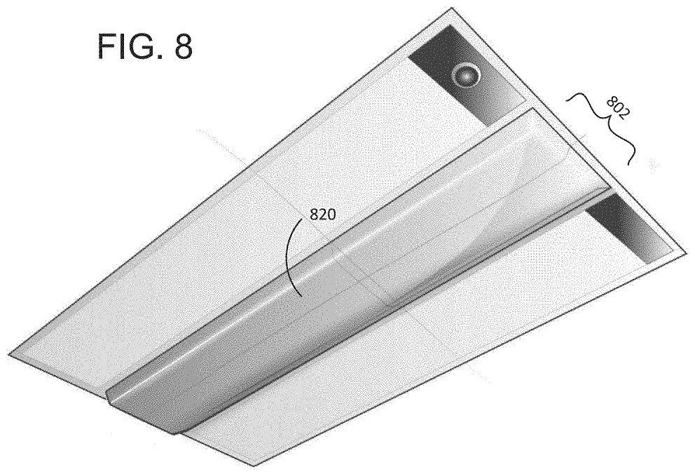

Thus, FIGS. 6A and 6B; and 7A and 7B illustrate fixture modules 601, 701 retaining the ultraviolet sources 204 and back cover arrangements 620, 720 and rotating upon respective longitudinal axes. FIGS. 6B and 7B illustrate ultraviolet sources 204 in their respective “storage” positions and with their respective back covers 620, 720 flush with the edges of their respective troffer light fixtures. FIG. 8 illustrates an alternative beveled back cover 820.

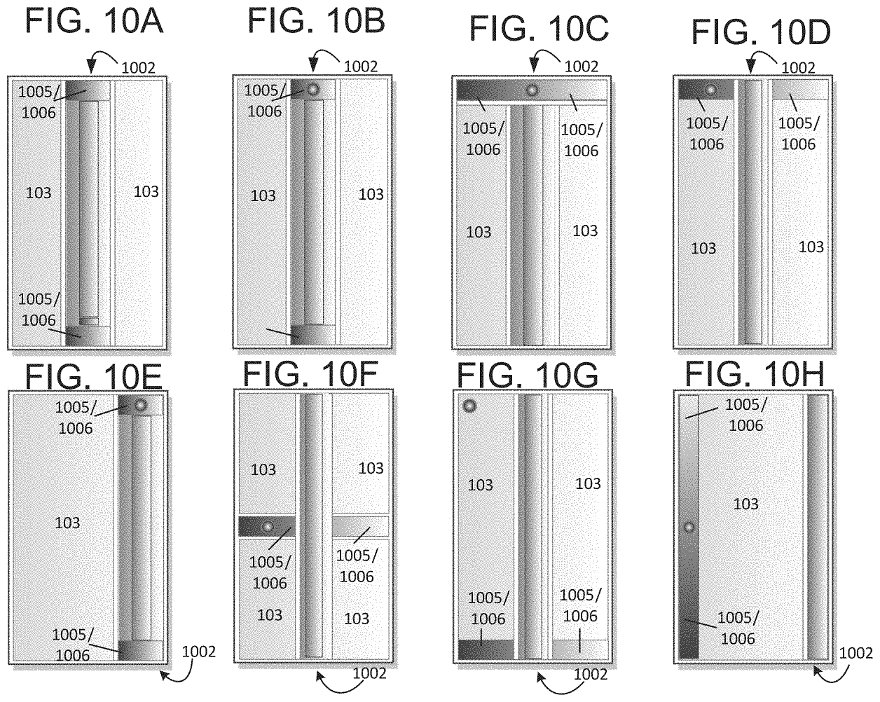

The disinfecting apparatus 202 of FIG. 2A is equipped with a pair of vents (205, 206) placed on opposite ends of the ultraviolet source 204. An intake vent 205 is positioned on one end of the ultraviolet source 204 and provides for the intake of room air into the disinfecting apparatus 202. An exhaust vent 206 is positioned on the opposite end of the ultraviolet source 204 and provides for the release of air from the disinfecting apparatus 202. While FIG. 2A illustrates the exhaust vent 206 on the same side as the hinge 203, the intake and exhaust vents 205, 206 may be arranged on either end (see FIG. 10 for a variety of arrangements). FIG. 11 is a cross-section of an exemplary disinfecting apparatus 1102 with a fixture module 1101 retaining and supporting an ultraviolet source 204. FIG. 11 illustrates the fixture module 1101 and ultraviolet source 204 rotated to the “storage” position, such that a back cover 1120 is visible. With the fixture module 1101 and ultraviolet source 204 rotated to the “storage” position, air drawn in the intake vent 205 is forced across the ultraviolet source 204 (and through the emitted ultraviolet light) to be exhausted via the exhaust vent 206 from the disinfecting apparatus 1102. Note that because the fixture module 1101 and associated ultraviolet source 204 are rotated to the “storage” position within the disinfecting apparatus 1102, ultraviolet light emitting from the ultraviolet source 1104 stays within the disinfecting apparatus 1102. FIG. 11 also illustrates an exemplary drive module 1142 that includes a gear box 1142a and a support fixture 1142b. The drive module 1142 rotates the fixture module 1101 (and associated ultraviolet source 204). As illustrated in FIG. 11, the drive module 1142 rotates the fixture module 1102 between the “storage” position and the “ready” position. Alternatively, as illustrated in FIG. 4, the drive module 1142 includes a hinge 203 upon which the ultraviolet source 204 is rotated from the “ready” position to the “deployed” position. The drive module 1142 includes any necessary hardware (e.g., a motor and associated gearing) to raise and lower, and/or, rotate the fixture module 1101 and associated ultraviolet source 204. In a further alternative embodiment, the disinfecting apparatus is in a “static” position and does not include a drive module, such that the support fixture and ultraviolet light are held in the “ready” position.

As illustrated in FIGS. 2A and 11, when the ultraviolet source 204 is energized while in the “storage” position (e.g., when instructed by the control module 1210 of FIG. 12), air forced through the disinfecting apparatus 202, 1102 will be exposed to the ultraviolet light and disinfected. Thus, the ultraviolet source 204 may be periodically energized to disinfect air that is drawn into the disinfecting apparatus 202, 1102. Running the ultraviolet source 204 to disinfect room or area air may be performed periodically or continuously while in the “storage” position. Note that air may be forced or drawn into the disinfecting apparatus 202, 1102 by one or more fans 1130. While a single fan 1130 is illustrated in FIG. 11, other fan arrangements are possible. For example, one or more fans may be positioned to draw air into the intake vents 205. Optionally, one or more additional fans may be positioned at the exhaust vent 206. Fans may also be placed in other locations within the disinfecting apparatus 1102.

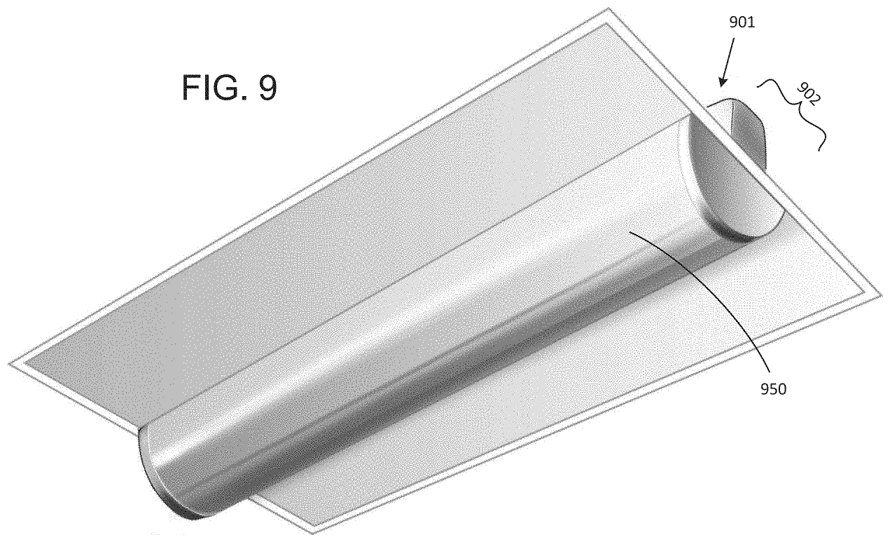

FIG. 9 illustrates a troffer light fixture (such as the troffer light fixture 100 of FIG. 1) with an alternative disinfecting apparatus 902 with a fixed cover 950 over a fixture module 901 that retains and supports an ultraviolet source 204. The fixture module 901 is configured to rotate the ultraviolet source 204 upon its longitudinal axis behind the fixed cover 950. That is, whether facing the room or area, or facing into the disinfecting apparatus 902, the ultraviolet source 204 is hidden behind the fixed cover 950. Note that the fixed cover 950 is configured to pass ultraviolet light through without substantially obstructing the light. FIGS. 10A-10H illustrate a variety of different configurations for positioning vents 1005, 1006 and light panels 103, with respect to their respective disinfecting apparatuses 1002.

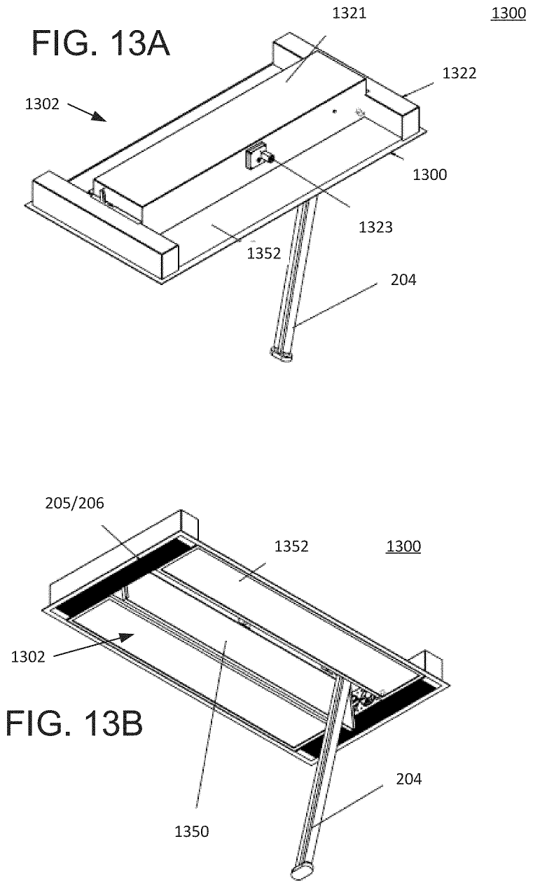

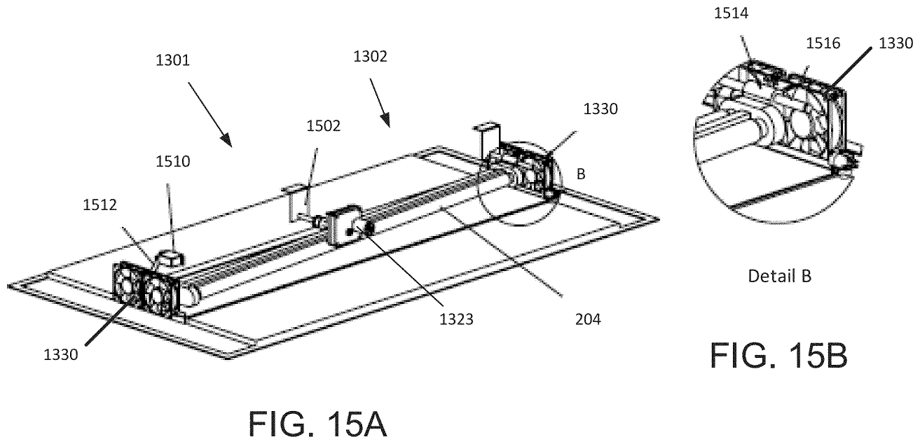

FIGS. 13A and 13B illustrate a troffer light fixture 1300 (such as the troffer light fixture 100 of FIG. 1) with an alternative disinfecting apparatus 1302 with a rotatable cover 1350 and a fixture module 1301 that retains and supports an ultraviolet source 204 (see FIGS. 15A and 15B). FIG. 13A illustrates an HVAC housing 1321 with a pair of HVAC inlet/outlet panels 1322 on either side. The fixture module 1301 rotates an adjustable light panel 1350 to expose the ultraviolet source 204. A pair of fixed light panels 1352 are arranged on either side of the adjustable light panel 1350. As illustrated in FIG. 13B, the fixture module 1301 also rotates the ultraviolet source 204 (via motor 1323) to extend the ultraviolet source 204 into a surrounding area under the disinfecting apparatus 1302. FIG. 13B illustrates the adjustable light panel 1350 in the open position between the fixed light panels 1352, and the ultraviolet source 204 extending from the disinfecting apparatus 1302.

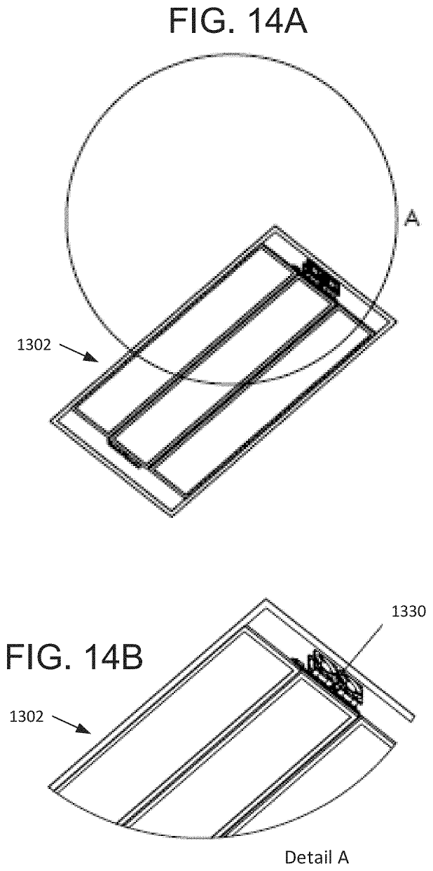

FIGS. 14A and 14B illustrate the adjustable light panel 1350 closed and concealing the ultraviolet source 204. FIGS. 14A and 14B also illustrate the position of the circulating fans 1330 on one side of the disinfecting apparatus 1302 and troffer light fixture 1300. FIG. 14B is a close-up of Detail A of FIG. 14A.

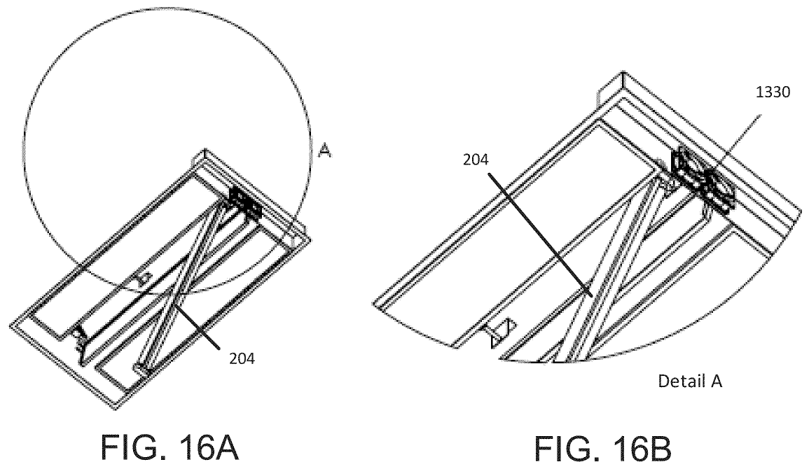

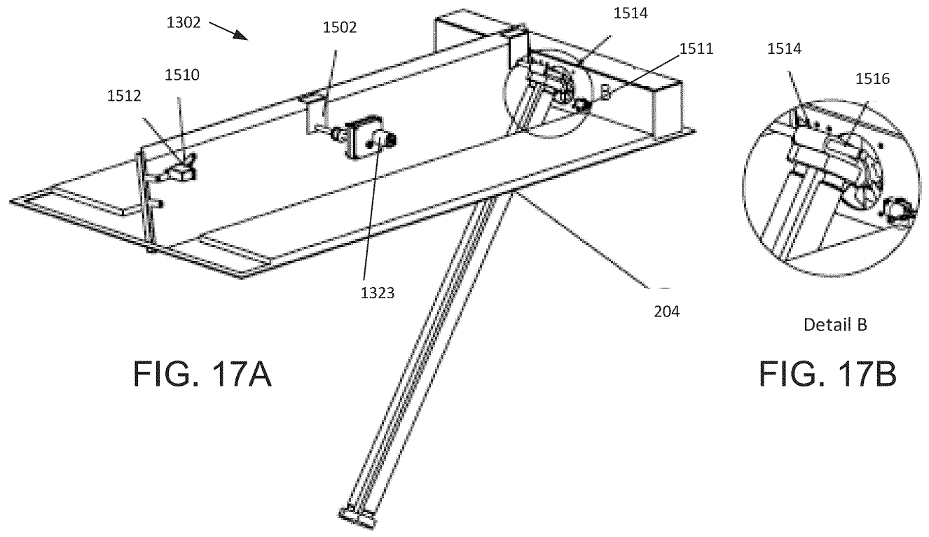

FIGS. 15A, 15B, 17A, and 17B illustrate several of the component parts of the fixture module 1301. For the sake of clarity, covers and some mechanical fixtures have been removed from the troffer light fixture 1300 and the fixture module 1301. FIGS. 15A, 15B, 17A, and 17B illustrate interior views of the fixture module 1301 and troffer light fixture 1330. While FIGS. 15A and 15B illustrate the ultraviolet source 204 retracted, FIGS. 17A and 17B illustrate the ultraviolet source 204 extended. FIGS. 16A and 16B, providing an exterior view of the underside of the troffer light fixture 1300, also illustrate the ultraviolet source 204 extended. A stability bracket 1502 is coupled to the motor 1323 to aid in lowering the ultraviolet source 204 (see FIG. 15A). FIG. 15A also illustrates a proximity sensor 1510 and a switch 1512, used to detect the position of the moving ultraviolet source 204, as well as optionally used to detect the presence of people below the disinfecting apparatus 1302. FIG. 15B is a close-up view of detail B that illustrates a bulb pivot mechanism 1514 and a pivot shaft 1516 which as discussed herein, are utilized in the lowering of the ultraviolet source 204. FIGS. 15A and 15B illustrate the circulating fans 1330 arranged on either side. FIG. 16B also provides a close-up view of the circulating fans 1330. FIG. 17A illustrates another view of the bulb pivot mechanism 1514 as well as an additional proximity sensor 1511.

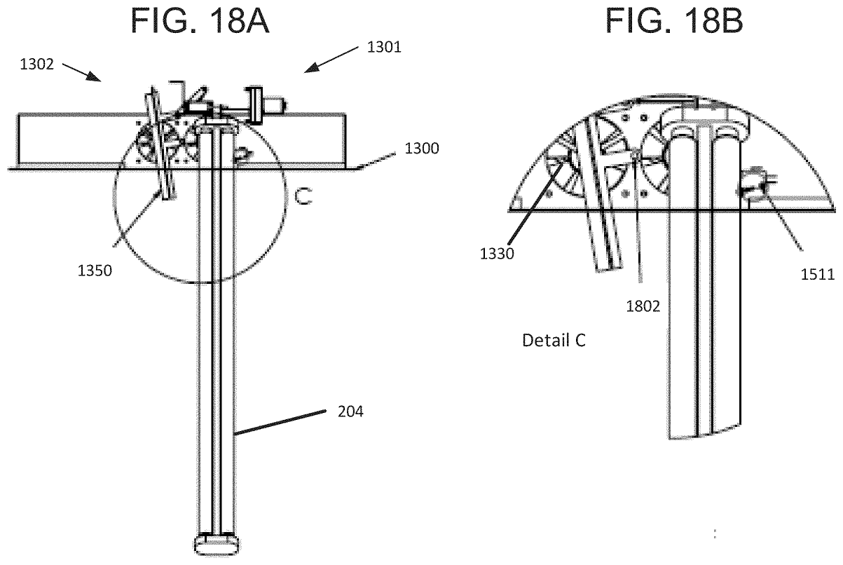

FIGS. 18A and 18B illustrate side views of the interior of the troffer light fixture 1300 and the disinfecting apparatus 1302. As illustrated in FIG. 18A, the adjustable light panel 1350 is open and the ultraviolet source 204 is extended from the disinfecting apparatus 1302. FIG. 18B illustrates a door pivot 1802 connected to the adjustable light panel 1350, as well as another view of the proximity sensor 1511.

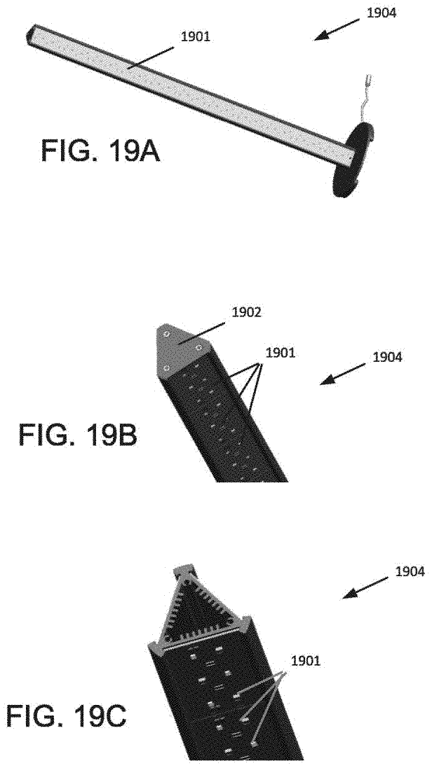

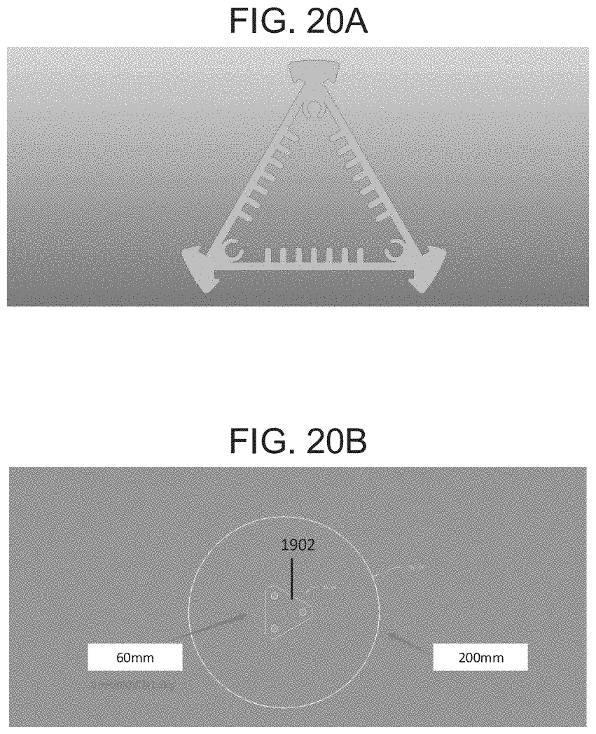

FIGS. 19 and 20 illustrate an exemplary ultraviolet source 1904. As illustrated in FIG. 19A, a shaft of the ultraviolet source 1904 is lined with LED UVC sources. As illustrated in FIGS. 19B and 19C, the ultraviolet source 1904 is arranged as a columnar triangular shape with three sets of LED UVC sources 1901 running down each of the sides of the ultraviolet source 1904. As illustrated in FIGS. 19B, 19C, and 20, the sides of the ultraviolet source 1904 are capped with triangular shaped, flat end caps 1902. As illustrated in FIGS. 19A and 20, the ultraviolet source 1904 may also include a flat disk 1902 affixed to an opposite end from the open end illustrated in FIG. 19C.

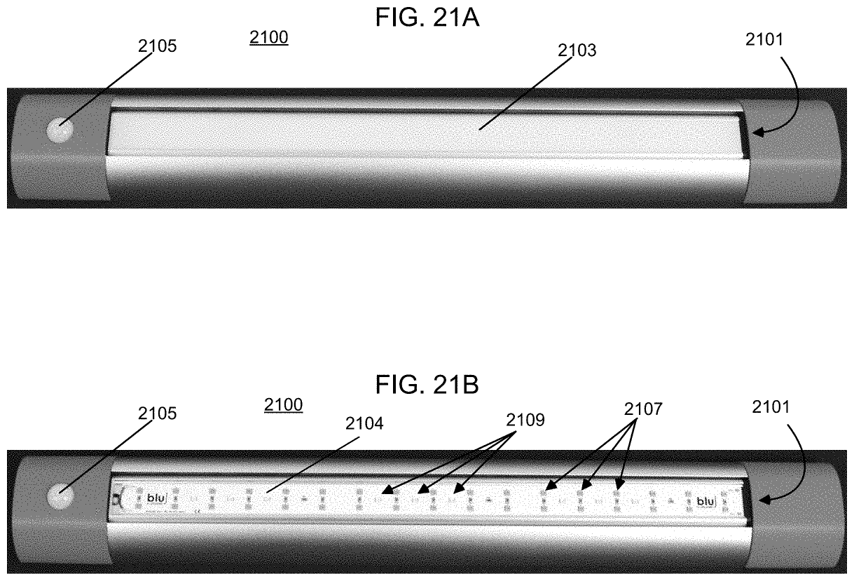

FIGS. 21-23 illustrate a rotatable disinfecting apparatus 2100 with an exemplary fixture module 2101, which retains and supports an ultraviolet source 2104 and a light panel 2103. The ultraviolet source 2104 and the light panel 2103 are arranged back-to-back within the fixture module 2101. The ultraviolet source 2104 and/or the light panel 2103 are selectively energized when they receive power through a wiring harness that is arranged within the fixture module 2101. The ultraviolet source 2104 and the light panel 2103 are energized with any of a variety of different power inputs, for example, 12 VDC, 24 VDC, 120 VAC, and 240 VAC. The fixture module 2101 rotates the ultraviolet source 2104 and the light panel 2103 about a longitudinal axis. As discussed herein, the fixture module 2101 includes mounting hardware and a motor for rotating the ultraviolet source 2104 and the light panel 2103. FIG. 21A illustrates the fixture module 2101 rotated about its longitudinal axis such that the light panel 2103 is flush with a housing of the rotatable disinfecting apparatus 2100 and facing a surrounding area or room. With the light panel 2103 exposed, as illustrated in FIG. 21A, the rotatable disinfecting apparatus 2100 can be used as a conventional lighting source. FIG. 21B illustrates the fixture module 2101 rotated about its longitudinal axis such that the ultraviolet source 2104 (which is opposite the light panel 2103) is flush with the housing of the rotatable disinfecting apparatus 2100 and facing the surrounding area or room. As in the other disinfecting apparatuses (602, 702, and 902) discussed herein (see FIGS. 6, 7, and 9), when the ultraviolet source 2104 has been rotated about its longitudinal axis to face the surrounding area or room, the ultraviolet source 2104 is in a “ready” position. Similarly, when the fixture module 2101 is rotated about its longitudinal axis to position the light panel 2103 flush with the rotatable disinfecting apparatus housing, the ultraviolet source 2103 is in a “storage” position.

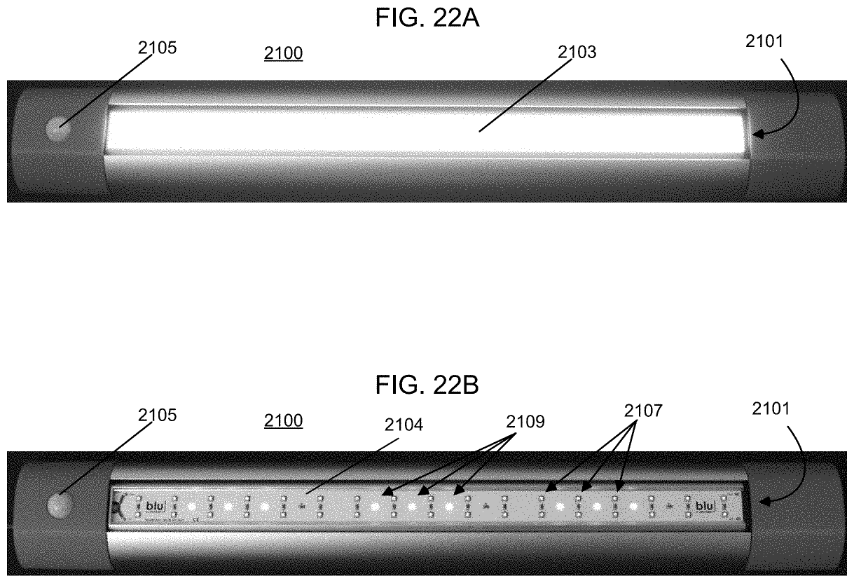

As illustrated in FIGS. 21-23, the rotatable disinfecting apparatus 2100 includes an occupancy detector 2105, which prevents the ultraviolet source 2104 from being energized while the surrounding area or room is occupied (see also FIG. 12). FIGS. 21B and 22B illustrate that the ultraviolet source 2104 includes an array of LED ultraviolet devices 2107, as well as a set of “tell-tale” lights 2109 that are illuminated when the individual ultraviolet devices 2107 of the ultraviolet source 2104 are energized. While the exemplary rotatable disinfecting apparatus 2100 includes LED ultraviolet devices 2107, the ultraviolet source 2104 may in other embodiments include one or more fluorescent light fixtures, excimer light fixtures, incandescent light fixtures, which may be arranged as one or more linear ultraviolet light fixtures or arranged in linear arrays of ultraviolet light fixtures.

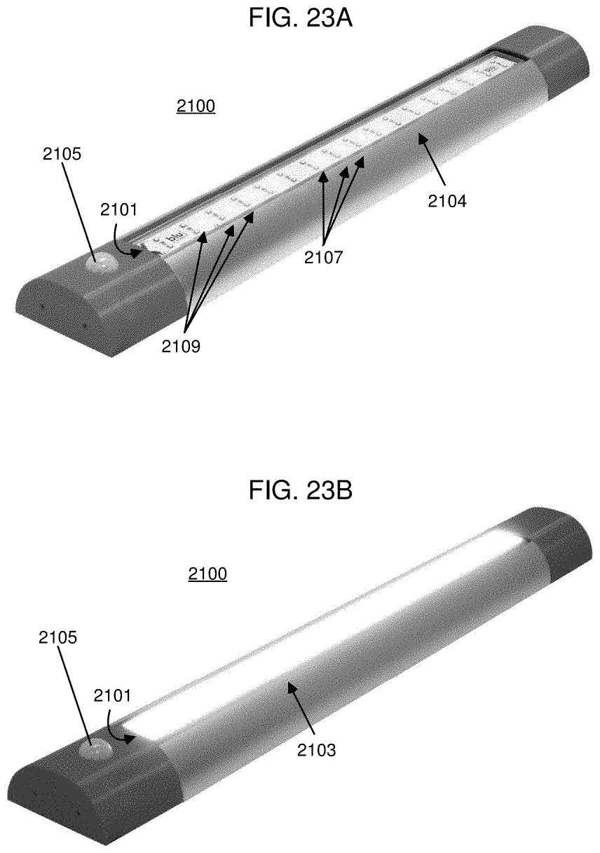

FIGS. 21A and 21B depict the light panel 2103 and the ultraviolet source 2104 (on the opposing side) de-energized (turned off), respectively. FIGS. 22A and 22B depict the light panel 2103 and the ultraviolet source 2104 energized, respectively. FIGS. 23A and 23B provide additional perspective views of the exemplary rotatable disinfecting apparatus 2100.

The rotatable disinfecting apparatus 2100 may be mounted on a ceiling or wall, inside a cabinet, or mounted to a vehicle, trailer or RV cross member. In other words, the rotatable disinfecting apparatus 2100 is configured for a variety of different mounting needs. The rotatable disinfecting apparatus 2100 may be controlled via RF remote control or alternatively via Wi-Fi functionality. In a further embodiment, the rotatable disinfecting apparatus 2100 may be hardwired for direct control of its functionality. As discussed herein, the rotatable disinfecting apparatus 2100 is configured to control the operation of the fixture module 2101 (and the ultraviolet source 2104 and the light panel 2103) via the wiring harness of the fixture module 2101.

Thus, multi-functional disinfecting apparatuses may be arranged in troffer light fixtures, mounted on walls or ceiling surfaces, or other surfaces, i.e., cabinet, vehicle, and/or trailer interiors, and mounted on crossbars and/or other supporting members. The disinfecting apparatus may be placed in a first orientation that exposes an ultraviolet source to a surrounding area or room (i.e., the “ready” position). While in the “ready” position, the ultraviolet source emits ultraviolet light that disinfects exposed surfaces within that surrounding area or room that are reached by the ultraviolet light. While in the “ready” position, the ultraviolet source may be optionally rotated on one end to extend down into the surrounding area or room, such that the emitted ultraviolet light more effectively reaches the exposed horizontal and vertical surfaces within the surrounding area or room. The disinfecting apparatus may also be placed in a second orientation that faces the ultraviolet source away from the surrounding area or room and into the disinfecting apparatus (i.e., the “storage” position). While in the “storage” position, even if the ultraviolet source is emitting ultraviolet light, the emitted ultraviolet light is shaded from the surrounding area or room. Instead, via a pair of vents arranged on opposite ends of the disinfecting apparatus, air may be drawn into the disinfecting apparatus to be disinfected as it passes through the disinfecting apparatus and then released back into the surrounding area or room. One or more fans may be positioned within the disinfecting apparatus to mechanically draw the air into the disinfecting apparatus and to force the air through the disinfecting apparatus.

Changes and modifications in the specifically described embodiments can be carried out without departing from the principles of the present invention which is intended to be limited only by the scope of the appended claims, as interpreted according to the principles of patent law including the doctrine of equivalents.

1. A system for disinfecting exposed surfaces and air passing through the system, the system comprising:
a disinfecting apparatus positioned above a space to be disinfected, the space comprises a surrounding area or room with exposed surfaces, the disinfecting apparatus comprising:
an ultraviolet source configured to emit ultraviolet light for disinfecting the exposed surfaces of the space;
a fixture module configured to retain and support the ultraviolet source within the disinfecting apparatus;
a drive module configured to move the fixture module and the ultraviolet source between a ready position and a storage position;
wherein the ultraviolet source is configured to emit ultraviolet light onto the exposed surfaces when in the ready position;
wherein the ultraviolet source is shielded from the surrounding area or room when in the storage position, such that emitted ultraviolet light will not reach the exposed surfaces; and
wherein the disinfecting apparatus is operable to disinfect air passing through the disinfecting apparatus when the ultraviolet source is in the storage position.

2. The system of claim 1, wherein the disinfecting apparatus further comprises a pair of vents arranged on opposite ends of the disinfecting apparatus, the pair of vents comprising an inlet vent for drawing air into the disinfecting apparatus and an exhaust vent for releasing the air from the disinfecting apparatus.

3. The system of claim 2, wherein the disinfecting apparatus comprises at least one fan, and wherein the at least one fan is positioned within the disinfecting apparatus to mechanically draw air into the disinfecting apparatus and to force the air through the disinfecting apparatus and through the ultraviolet light emitted by the ultraviolet source when in the storage position.

4. The system of claim 1, wherein the emitted ultraviolet light is ultraviolet C light.

5. The system of claim 1, wherein the ultraviolet source comprises one or more ultraviolet light emitting devices including one or more incandescent bulbs or light fixtures, excimer light fixtures, fluorescent bulbs or light fixtures, and light emitting diodes, wherein the incandescent bulbs/light fixtures are operable to emit one of 222 nm or 254 nm ultraviolet light, wherein the fluorescent bulbs/light fixtures are operable to emit one of 222 nm or 254 ultraviolet light, wherein the excimer light fixtures are operable to emit 222 nm ultraviolet light, and wherein the light emitting diodes are operable to emit 254 nm ultraviolet light.

6. The system of claim 1, wherein the drive module is configured to rotate the ultraviolet source about one end when the ultraviolet source is in the ready position within the fixture module, to extend the ultraviolet source into the surrounding area or room such that the ultraviolet source is perpendicular to the disinfecting apparatus, and wherein when perpendicular to the disinfecting apparatus, the ultraviolet source is in a deployed position.

7. The system of claim 1 comprising a control module and a proximity sensor, wherein the control module is configured to control the operation of the disinfecting apparatus such that the drive module retains or returns the ultraviolet source to the storage position within the fixture module when a person is detected by the proximity sensor, and wherein the control module is configured to control the operation of additional disinfecting apparatuses arranged to emit ultraviolet light on the exposed surfaces of the surrounding area or room.

8. The system of claim 7, wherein the control module is operable to direct the drive module to position the ultraviolet source in the ready position for a selected period of time so long as no person is detected by the proximity sensor.

9. The apparatus of claim 1, wherein the drive module is configured to move the ultraviolet source between the ready position and the storage position by rotating the ultraviolet source along a longitudinal axis within the fixture module, wherein the drive module is configured to move the ultraviolet source between the ready position and the deployed position by rotating the ultraviolet source about a hinge on one end of the ultraviolet source, and wherein the fixture module is configured to remove a cover from the ultraviolet source when the ultraviolet source is positioned in the ready position and to restore the cover when the ultraviolet source is positioned in the storage position.

10. The system of claim 1, wherein the disinfecting apparatus is configured for mounting within one of:
a standalone housing unit configured to retain and support the disinfecting apparatus above the space; and
a troffer light fixture configured to retain and support the disinfecting apparatus above the space.

11. A method for sanitizing exposed surfaces and air passing through a sanitizing apparatus, the method comprising:
emitting ultraviolet light from an ultraviolet source of the sanitizing apparatus mounted above a space to be sanitized, wherein the ultraviolet light is operable to sanitize the exposed surfaces of the space, and wherein the space comprises a surrounding area or room;
placing the ultraviolet source into a ready position such that the emitted ultraviolet light can reach exposed surfaces of a surrounding area or room;
placing the ultraviolet source into a storage position such that emitted ultraviolet light will not reach the exposed surfaces of the surrounding area or room; and
drawing air into the sanitizing apparatus while the ultraviolet source is in the storage position such that the drawn air is passed through the ultraviolet light to sanitize the air.

12. The method of claim 11, wherein drawing air into the sanitizing apparatus comprises:
drawing air into an inlet vent of the disinfecting apparatus;
passing the air through the disinfecting apparatus; and
expelling the air from the disinfecting apparatus through an exhaust vent of the disinfecting apparatus.

13. The method of claim 12, wherein the air is drawn into the disinfecting apparatus via at least one fan, and wherein the at least one fan is positioned within the disinfecting apparatus to mechanically draw the air into the disinfecting apparatus and to force the air through the disinfecting apparatus and through the ultraviolet light emitted by the ultraviolet source when in the storage position.

14. The method of claim 11, wherein the emitted ultraviolet light is ultraviolet C light.

15. The method of claim 11, wherein the ultraviolet source comprises one or more ultraviolet light emitting devices including one or more incandescent bulbs or light fixtures, excimer light fixtures, fluorescent bulbs or light fixtures, and light emitting diodes, wherein the incandescent bulbs/fixtures emit one of 222 nm and 254 nm ultraviolet light, wherein the fluorescent bulbs/light fixtures emit one of 222 nm and 254 nm ultraviolet light, wherein the excimer light fixtures emit 222 nm ultraviolet light, and wherein the light emitting diodes emit 254 nm ultraviolet light.

16. The method of claim 11 further comprising rotating the ultraviolet source when the ultraviolet source is in the ready position, to extend the ultraviolet source into the surrounding area or room such that the ultraviolet source is perpendicular to the disinfecting apparatus, and wherein when perpendicular to the disinfecting apparatus, the ultraviolet source is in a deployed position.

17. The method of claim 11 further comprising controlling the operation of the disinfecting apparatus with a proximity sensor, such that the ultraviolet source will be retained or returned to the storage position when a person is detected by the proximity sensor, and further comprising controlling the operation of additional disinfecting apparatuses to selectively emit ultraviolet light on the exposed surfaces of the surrounding area or room.

18. The method of claim 17 further comprising directing the disinfecting apparatus to position the ultraviolet source in the ready position for a selected period so long as no person is detected by the proximity sensor.

19. The method of claim 11, wherein moving the ultraviolet source between the ready position and the storage position comprises rotating the ultraviolet source along a longitudinal axis, wherein the disinfecting apparatus is configured to move the ultraviolet source between the ready position and the deployed position by rotating the ultraviolet source about a hinge on one end of the ultraviolet source, and wherein the disinfecting apparatus removes a cover from the ultraviolet source when the ultraviolet source is positioned in the ready position and restores the cover when the ultraviolet source is positioned in the storage position.

20. The method of claim 11, wherein the disinfecting apparatus is mounted within one of:
a standalone housing unit that retains and supports the disinfecting apparatus above the space; and
a troffer light fixture that retains and supports the disinfecting apparatus above the space.
